(19) United States
(12) Patent Application Publication
(10) Pub. No.: US 2021/0244876 A1
(43) Pub. Date: Aug. 12, 2021

(54) MEDICAL CANNULA PACKAGE

(71) Applicant: Becton, Dickinson and Company, Franklin Lakes, NJ (US)

(72) Inventors: Amit Limaye, Wayne, NJ (US); David Poganski, Midland Park, NJ (US); Aditya Prabhu, Harrison, NJ (US); Giridhar Shamsunder, Pittsboro, NC (US); Pearse Egan, Dublin (IE)

(73) Assignee: Becton, Dickinson and Company, Franklin Lakes, NJ (US)

(21) Appl. No.: 16/972,937

(22) PCT Filed: May 29, 2019
(86) PCT No.: PCT/US2019/034252

§ 371 Date: Dec. 7, 2020

Related U.S. Application Data
(60) Provisional application No. 62/682,532, filed on Jun. 8, 2018.

(51) Int. Cl. A61M 5/00; B65D 75/00; B65D 77/00; B65D 85/24; B65B 19/00

(52) U.S. Cl. CPC A61M 5/002; B65D 75/00; B65D 77/00; A61M 2207/00; B65B 19/00; A61M 2205/583; B65D 85/24

(57) ABSTRACT

A package (116) for a medical cannula, including a plurality of medical cannula outer shields (29), the outer shields each being configured for receiving a medical cannula therein. The package also includes a web (100) having a plurality of holes (104) therethrough corresponding to the plurality of outer shields. Each of the corresponding plurality of holes retains one of the plurality of medical cannula outer shields therein.

### CROSS-REFERENCE TO RELATED APPLICATION

This application is the U.S. national stage of International Application No. PCT/US2019/034252, filed May 29, 2019, which claims the benefit of U.S. provisional Application No. 62/682,532, filed Jun. 8, 2018, in the U.S. Patent and Trademark Office, the disclosures of which are incorporated herein by reference in its entirety.

### BACKGROUND OF THE INVENTION

### 1. Field of the Invention

The present invention relates to medical cannula packaging, and more particularly, to packaging for dispensing and storing a medical cannula.

### 2. Description of the Related Art

Medication delivery pens are used for self-injection of precisely measured doses of medication. Pens are widely used, for example, by diabetics to self-inject insulin. A typical medication delivery pen includes a cartridge that contains a volume of liquid medication sufficient for several doses. Using a disposable pen needle attached to the pen device, a user injects the dose into a tissue area, such as the intramuscular tissue layer, the subcutaneous tissue layer, or the intradermal tissue layer.

Pen injection devices, such as the exemplary pen injector 50 shown in FIGS. 1 and 2, typically comprise a dose knob/button 24, an outer sleeve 13, and a cap 21. The dose knob/button 24 allows a user to set the dosage of medication to be injected. The user typically grips the outer sleeve 13 when injecting medication. The cap 21 can securely hold the pen injector 50 in a shirt pocket, purse, or other suitable location.

FIG. 2 is an exploded view of the exemplary drug delivery pen 50 shown in FIG. 1. The dose knob/button 24 has a dual purpose and is used to both set the dosage of the medication to be injected and to inject the dosed medicament via a lead screw 7 and a stopper 15 from a medicament cartridge 12, which is attached to the drug delivery pen through a lower housing 17. The medicament cartridge 12 is typically a glass tube sealed at one end with a septum 16 and at the other end with the stopper 15. In standard drug delivery pens, the dosing and delivery mechanisms are all found within the outer sleeve 13. Those mechanisms are not described in greater detail herein because they are understood by one of ordinary skill in the art.

A medical cannula such as a pen needle assembly 10 includes a hub 20, a hollow patient needle 11 extending from a patient end of the pen needle assembly, and a septum-penetrating needle cannula 18 disposed within the hub 20 on a non-patient side thereof. The septum-penetrating needle cannula 18 is in fluid communication with the patient needle 11. The hub 20 is preferably threaded onto the lower housing 17, although other attachment means can be used such as attaching directly to the medicament cartridge 12. In attaching the hub 20 to the lower housing 17 or medicament cartridge 12, the septum-penetrating cannula 18 pierces the septum 16, but the septum 16 does not move with respect to the medicament cartridge 12. The stopper 15, however, is axially displaceable within the medicament cartridge 12 while maintaining a fluid-tight seal. The distal movement of the plunger or stopper 15 within the medicament cartridge 12 (due to advancement of the lead screw 7) causes medication to be forced into the patient needle 11 of the hub 20.

To protect a user, or anyone who handles the pen injector 50, pen needle assemblies are usually individually packaged inside a plastic cover with a peelable label covering the opening in the cover to provide a sterility barrier. For example, a rigid outer shield 29 attaches to and covers the hub 20. The outer shield 29 can also be used as a handle or grip to screw the hub 20 onto or off of the pen injector 50. Typically, a teardrop-shaped cover or label 32 provides a sterility barrier for the contents of the outer shield 29. The label 32 attaches to a top flange 30 of the outer shield 29 and has a tab 34 serving as a handle (shown in FIG. 5). An inner shield or needle cover 28 covers the patient needle 11 within the outer shield 29. The inner shield 28 can be secured to the hub 20 to cover the patient needle 11 by any suitable means, such as an interference fit or a snap fit. The outer shield 29 and inner shield 28 are removed prior to use. The cap 21 fits snugly against outer sleeve 13 to allow a user to securely carry the pen injection device 50.

Individually packaged pen needle assemblies are often sold packed loosely in a container, such as a box. Boxes of various sizes are used for various quantities of the individually packaged pen needle assemblies (for example, a 50-count box or a 100-count box).

### SUMMARY OF EMBODIMENTS OF THE INVENTION

Accordingly, it is an aspect of the present invention to provide improved packaging for pen needle assemblies.

The foregoing and/or other aspects of the present invention are achieved by a package for a medical cannula, including a plurality of medical cannula outer shields, the outer shields each being configured for receiving a medical cannula therein. The package also includes a web having a plurality of holes therethrough corresponding to the plurality of outer shields. Each of the corresponding plurality of holes retains one of the plurality of medical cannula outer shields therein.

The foregoing and/or other aspects of the present invention are also achieved by a method of packaging a plurality of medical cannulas, including providing a plurality of medical cannula outer shields, each having an opening therein, the outer shields each being configured for receiving one of the plurality of medical cannulas therein, and respectively inserting the plurality of medical cannulas into the plurality of outer shields. The method also includes providing a web having a plurality of holes therethrough corresponding to the plurality of outer shields, respectively inserting the plurality of outer shields into the corresponding plurality of holes to secure the outer shields in the web, and providing a sterility barrier for the outer shields with the medical cannula disposed therein.

Additional and/or other aspects and advantages of the present invention will be set forth in the description that follows, or will be apparent from the description, or may be learned by practice of the invention.

### BRIEF DESCRIPTION OF THE DRAWINGS

The above and/or other aspects and advantages of embodiments of the invention will become apparent and more readily appreciated from the following detailed description, taken in conjunction with the accompanying drawings, in which:

FIG. 1 is a perspective view of an exemplary drug delivery pen;

FIG. 2 is an exploded view of the exemplary drug delivery pen of FIG. 1;

FIGS. 3 and 4 are perspective views of a pen needle assembly that can be used in embodiments of the present invention;

FIG. 5 is a perspective view of an outer shield for the pen needle assembly of FIGS. 3 and 4;

FIG. 6 is a plan view of a web in accordance with an embodiment of the present invention;

FIGS. 7 and 8 are respective perspective views of a web unit and an outer shield inserted in a web unit in accordance with an embodiment of the present invention;

FIG. 9 illustrates a process of applying a sterility barrier in accordance with an embodiment of the present invention;

FIGS. 10-12 are respective side, top perspective, and bottom perspective views of the outer shield and a web unit of FIG. 8 with a sterility barrier in accordance with an embodiment of the present invention;

FIGS. 13 and 14 illustrate a series of marking in accordance with an embodiment of the present invention;

FIG. 15 illustrates a nested pair of packages in accordance with an embodiment of the present invention;

FIGS. 16-19 illustrate outer packaging in accordance with embodiments of the present invention;

FIGS. 20 and 21 illustrate a horizontally oriented tray package;

FIGS. 22 and 23 illustrate an angled tray package;

FIGS. 24-27 illustrate vertically oriented tray packages; and

FIGS. 28 and 29 illustrate embodiments of punch-through or pierce-through sterility barriers.

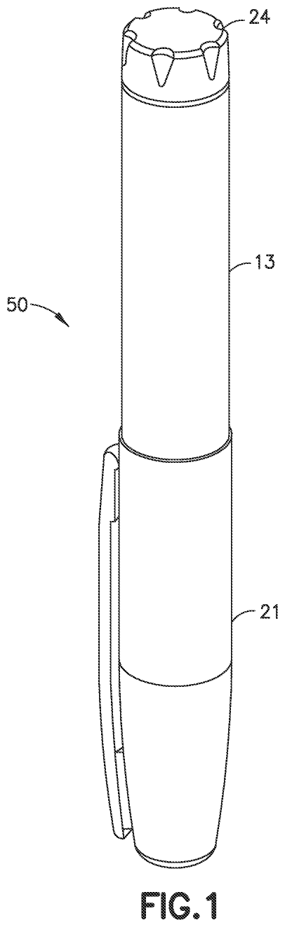

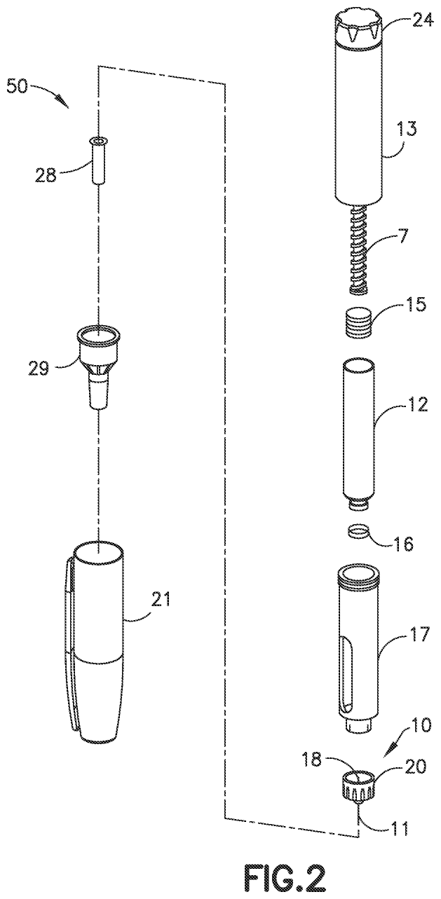

### DETAILED DESCRIPTION OF EMBODIMENTS OF THE PRESENT INVENTION

Reference will now be made in detail to embodiments of the present invention, which are illustrated in the accompanying drawings, wherein like reference numerals refer to like elements throughout. The embodiments described herein exemplify, but do not limit, the present invention by referring to the drawings.

It will be understood by one skilled in the art that this disclosure is not limited in its application to the details of construction and the arrangement of components set forth in the following description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled”” and variations thereof are not restricted to physical or mechanical connections or couplings. Further, terms such as “up,” “down,” “bottom,” “top,” “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges around and including the given value, for example, general tolerances associated with manufacturing, assembly, and use of the described embodiments.

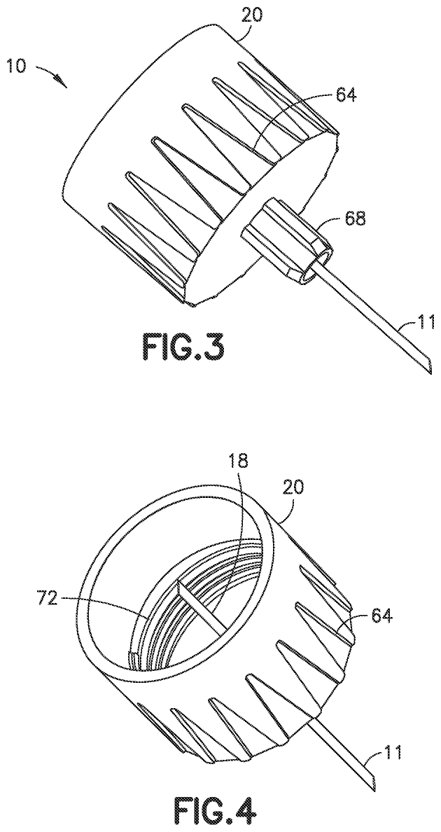

FIGS. 3 and 4 are perspective views of a pen needle assembly 10 that can be used in embodiments of the present invention. For brevity, the phrase “pen needle 10” will be used hereinafter instead of “pen needle assembly 10.” As shown in FIG. 3, the pen needle 10 includes a plastic hub 20 disposed at a non-patient end thereof. The hub 20 includes a plurality of ribs 64 for engagement with anti-rotation/retaining structures that will be described in greater detail below. In addition, protrusion 68 extends from a patient end of the hub 20 and the hollow patient needle 11 extends from the protrusion 68. The septum-penetrating metal needle cannula 18 (best shown in FIG. 4) disposed within the non-patient end of the hub 20 fluidly communicates with the patient needle 11. Preferably, the septum-penetrating metal needle cannula 18 and the hollow patient needle 11 are the same hollow needle sharpened at both ends.

Further, as shown in FIG. 4, the interior of the non-patient end of the hub 20 includes threads 72 for connection with an injection device, such as the pen injector 50. For brevity, hereinafter, the pen injector 50 will be employed as an exemplary injection device. One skilled in the art, however, will appreciate that other types of injection devices may be used without departing from the scope of the present invention. FIG. 5 is a perspective view of a typical outer shield 29 for the pen needle assembly of FIGS. 3 and 4.

FIG. 6 is a plan view of a web 100 in accordance with an embodiment of the present invention. The web 100 and provide a structure within which pen needle outer covers, e.g., outer shields 29 can be housed or nested and retained. The web 100 includes a plurality of web units 102 separated by perforations. The perforations between adjacent web units 102 facilitate separation of individual web units 102 from the web 100 or separation of groups of web units to form a smaller web 100 (or sub cluster or sub web, e.g., as illustrated in FIG. 6 as 112).

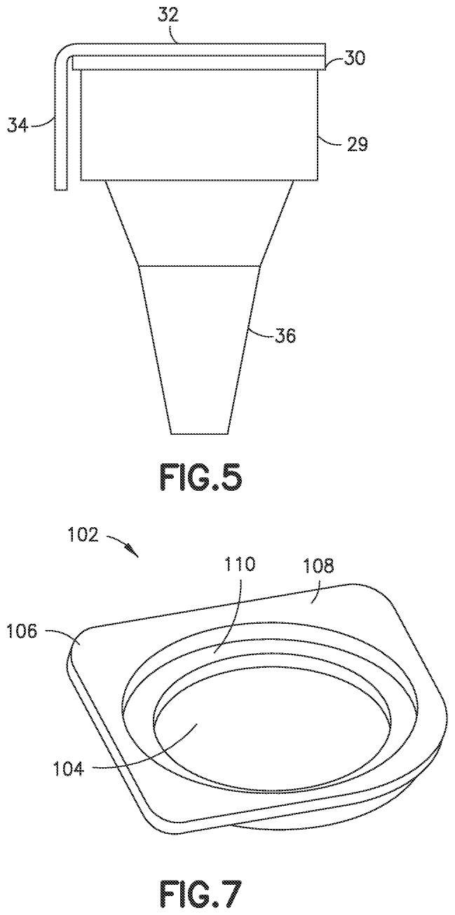

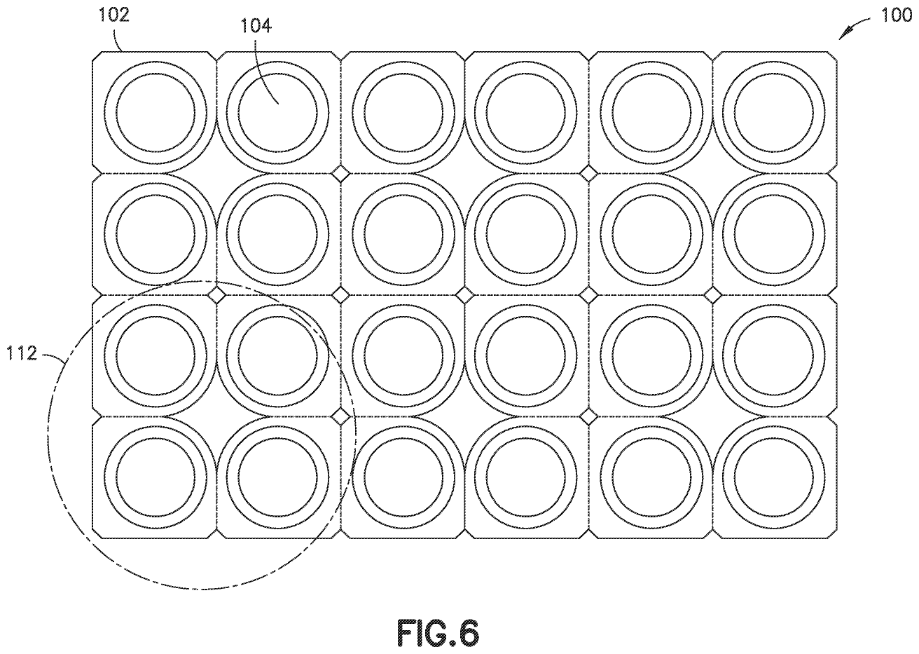

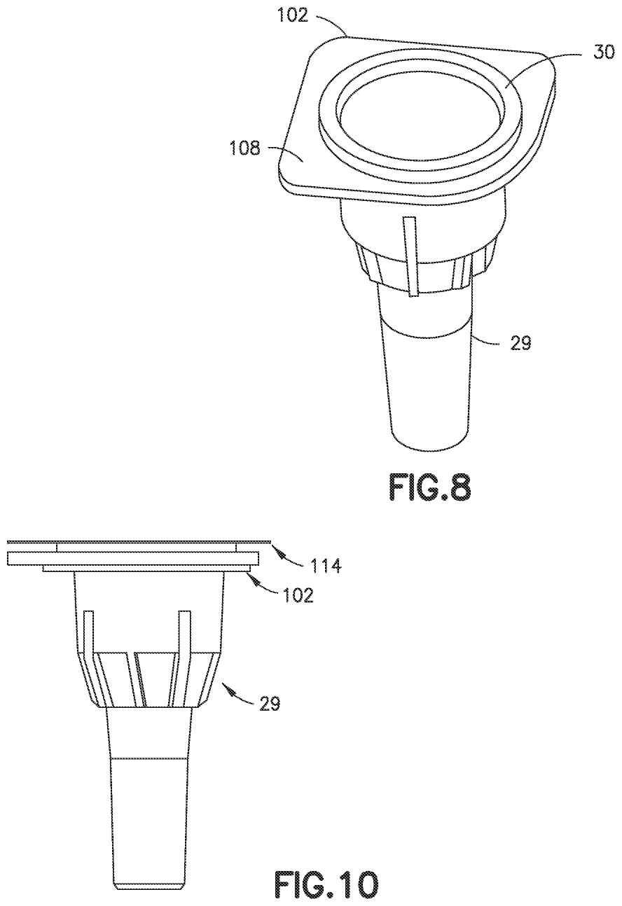

Each web unit 102 (see FIG. 7) has a hole 104 therethrough, and a web flange 106 surrounding the hole 104. Preferably, each web flange 106 includes a proximal flange 108 and a recessed flange 110. According to one embodiment, the recessed flange 110 is configured to receive a flange 30 of an outer shield 29, as shown in FIG. 8 when the outer shield 29 is securely received or retained in the web unit 102, e.g., via friction fit or interference fit or snap fit.

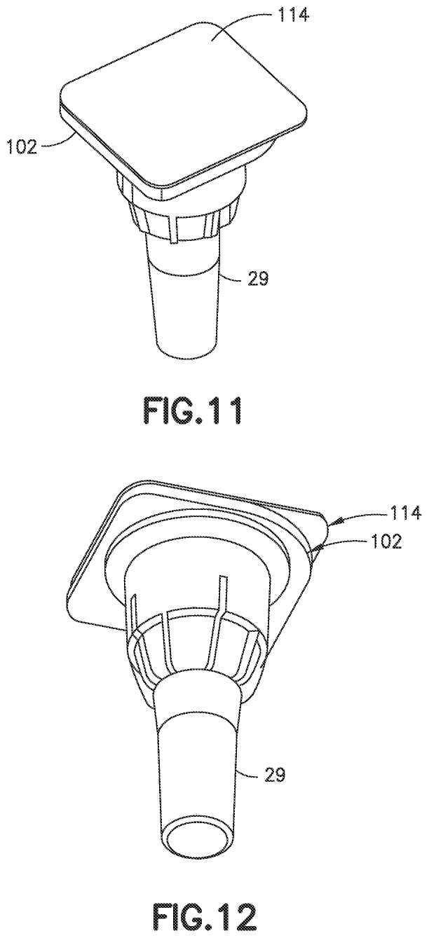

Referring to FIGS. 6-12, after a manufacturer or other user inserts a plurality of outer shields 29 through the holes 104 and secures or retains the outer shields 29 in the web 100, the manufacturer or other user can apply a removable sterility barrier or peel label 114. The pen needles 10 can be inserted into the outer shields 29 either before or after securing the outer shields 29 in the web 100, although insertion of the pen needles 10 into the outer shields 29 preferably occurs prior to installation of the ouster shields into the web 100. Sterilization preferably occurs subsequent to application of the sterility barrier 114, but can occur prior to application of the sterility barrier 114, as understood by those skilled in the art.

The web 100, the outer shield 29, and the hub 20 can be made of plastic, such as polypropylene (PP), polyethylene (PE), polycarbonate (PC), acrylonitrile butadiene styrene (ABS), and polyether ether ketone (PEEK). Different components can be made of different plastics. The sterility barrier 114 can be made of many different materials, such as plastic, paper, foil, or a combination of foil and paper.

The configuration and shape of the package 116 achieve the sealing and maintenance of sterility at the individual pen needle level. For example, as shown in FIG. 10, according to one embodiment, the proximal flange 30 of the outer shield 29 is disposed proximal to the web unit 102, while a portion of the outer shield 29 is disposed distally of the web unit 102. In this embodiment, the sterility barrier 114 removably adheres directly to the outer shield 29. According to one embodiment, the sterility barrier 114 removably adheres only to the outer shield 29. According to another embodiment, the depth of the recessed flange 110 is configured so that although the proximal flange 30 is proximal of the proximal surface of the web 100, when the sterility barrier 114 is applied, the sterility barrier 114 removably adheres to both the outer shield 29 and the proximal surface of the web 100.

According to yet another embodiment, the recessed flange 110 is configured to receive the outer shield 29 so that the proximal surface of the proximal flange 30 is substantially flush with the web 100 (web unit 102). In this embodiment, the sterility barrier 114 also removably adheres to both the outer shield 29 and the proximal surface of the web 100. In a further embodiment, the recessed flange 110 is sufficiently deep that the proximal flange is disposed distally of the proximal surface of the web, and in this embodiment, the sterility barrier 114 removably adheres only to the proximal surface of the web 100.

The end user exposes the sterile pen needle 10 for attachment to a pen injector 50 by removing the sterility barrier 114. Referring to FIGS. 6, 7, 9, and 12, according to one embodiment, each web unit 102 has three corners where the proximal flange 108 has a greater surface area than the fourth, more rounded corner. This configuration provides a grip for easily removing the sterility barrier 114, as shown in FIG. 12. In other words, the size and shape of the sterility barrier 114 are different from those of the web unit 102. This provides a gripping surface for the user to access the pen needle inside the outer shield 29.

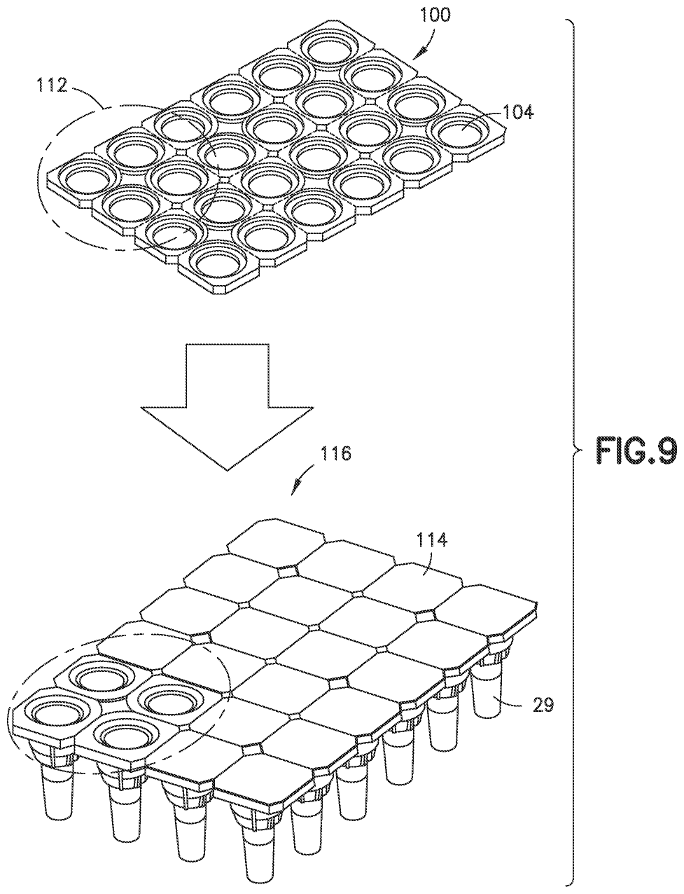

Orienting the web units 102 so that these respective fourth, more rounded corners are adjacent in the web 100 (or sub cluster or sub web, e.g., as illustrated in FIG. 6 as 112) permits the use of less material when applying the sterility barrier 114, as shown in FIG. 9, thereby reducing the overall cost of producing the package 116.

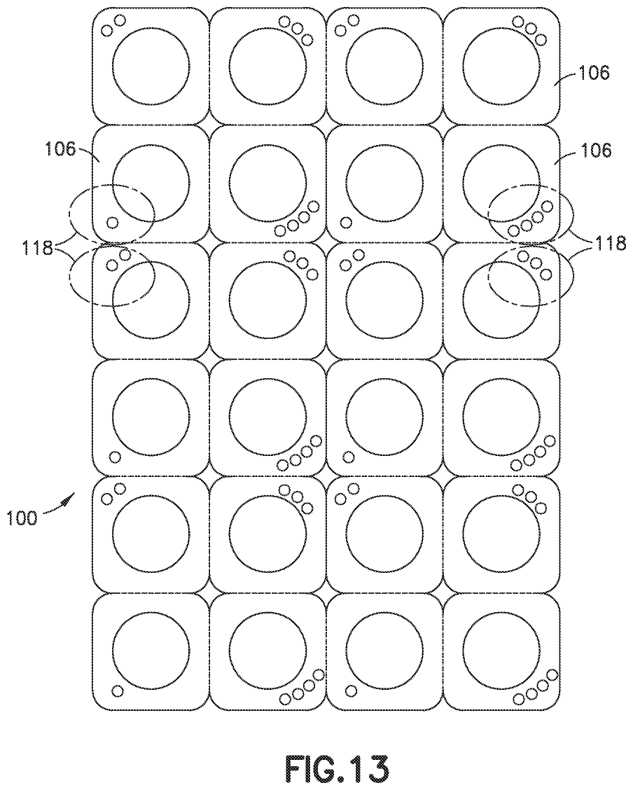

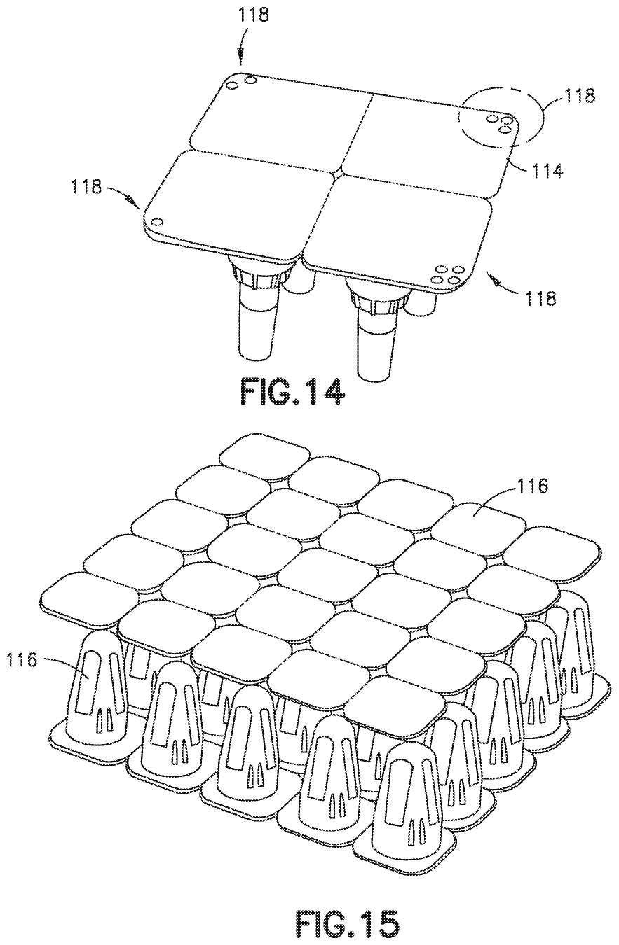

It is known that injection site rotation is beneficial for diabetes patients because repeat injection in a given area can lead to lipohypertrophy. According to embodiments of the present invention, as shown in FIGS. 13 and 14, the web flange 106, the sterility barrier 114, or both the web flange 106 and the sterility barrier 116 of each web unit 102 can include a marking 118 of a series of markings to suggest site rotation to the end user. For example, as shown in FIGS. 13 and 14, the markings 118 can be a series of dots. More specifically, in these embodiments, each web unit 102 and/or sterility barrier 114 is marked with from one to four dots, signifying four different injections sites, for example, arm, thigh, abdomen, and buttock.

The markings 118 can be a bump, texture, ink mark, or other marking without departing from the scope of the present invention. For example, the markings 118 could be numbers, such as Arabic or Roman numerals. Although in the shown embodiments, the series of markings 118 includes four members, one skilled in the art will appreciate that the series of markings 118 can have fewer or more than four members without departing from the present invention's scope.

As shown in FIGS. 13 and 14, the markings 118 are laid out in a two by two matrix of the web 100/package 116. One skilled in the art will appreciate, however, that the layout of the markings with respect to the web 100/package 116 can differ without departing from the scope of the present invention.

While these embodiments do not ensure that patients will use different sites/locations for consecutive injections, they provide a visual and/or tactile cue that can be associated with a certain site, and can aid users in site rotation.

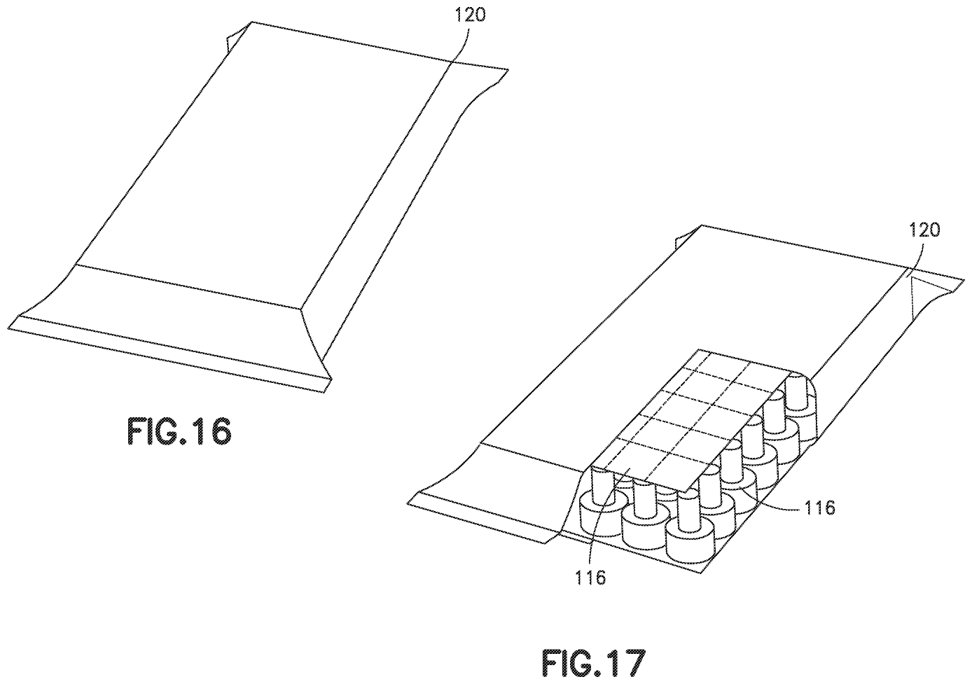

FIG. 15 illustrates a pair of nested packages 116 in accordance with an embodiment of the present invention. As shown in FIG. 15, because of the structure and spacing of the packages 116 employing webs 100, embodiments of the present invention provide for a more structured packing, allowing for both improved packing efficiency, and improved stackability. These improvements provide for more efficient outer packaging or shipping packaging, as shown in FIGS. 16-19.

FIGS. 16 and 17 illustrate a flow wrap or foil outer packaging 120 enclosing a nested pair of packages 116. This outer packaging configuration is expected to be more portable compared to, for example a cardboard box configuration, and may also be easier to use. The pouches of flow wrap or foil outer packaging 120 are expected to be capable of being stacked in a load bearing direction and thus, can be either packaged at intermediate count levels or bulk packed directly onto pallets for shipping, thereby significantly reducing additional packaging material and handling costs.

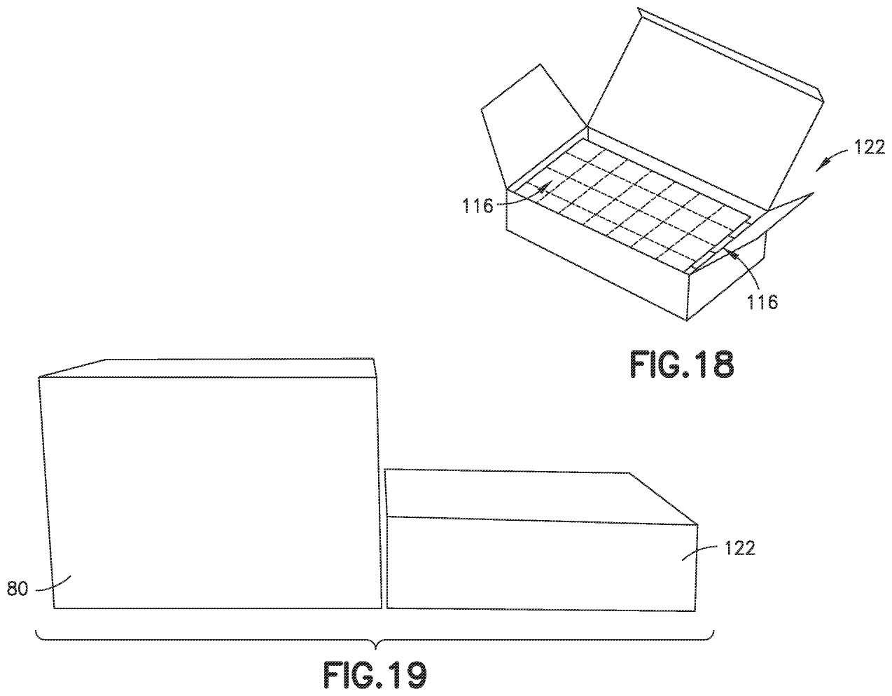

But cardboard box configurations of outer packaging can also be employed and still realize cost savings. FIG. 18 illustrates a box carton outer packaging 122 housing a nested pair of packages 116, and FIG. 19 shows a comparison between a currently employed loose package box of 100 sealed outer shields 29 and the box carton outer packaging 122 enclosing the same number of outer shields nested in packages 16 employing webs 100.

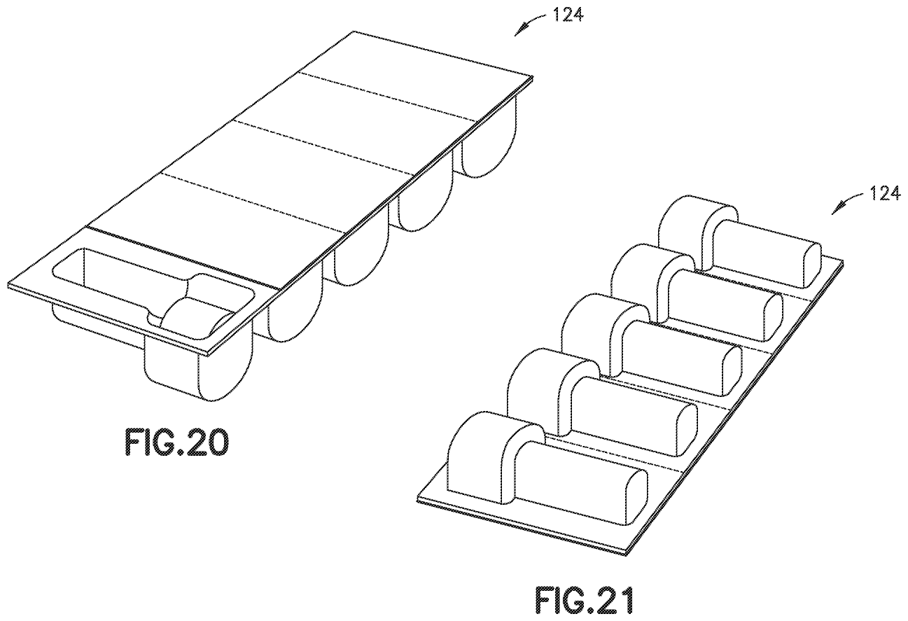

Nestable packages of pen needles and/or other medical sharps can also be made by thermoforming plastic or otherwise forming by controlled deformation of a starting component such as a plastic sheet. In instances in which material properties limit the geometry of the shapes (an example would be wherein the ratio of the area of material to draw from to the draw depth is too small thereby causing excessively thin walls, or puncture of the drawn pocket) that can be formed using commonly used plastics, an alternative approach can be used to obtain tray functionality. FIGS. 20 and 21 illustrate a horizontally oriented tray package 124 that horizontally houses an array of pen needles 10 or other medical sharps. Preferably, the horizontally oriented tray package 124 has perforations separating adjacent pen needles 10, so that individual packaged pen needles or smaller arrays can be easily formed for the user's convenience.

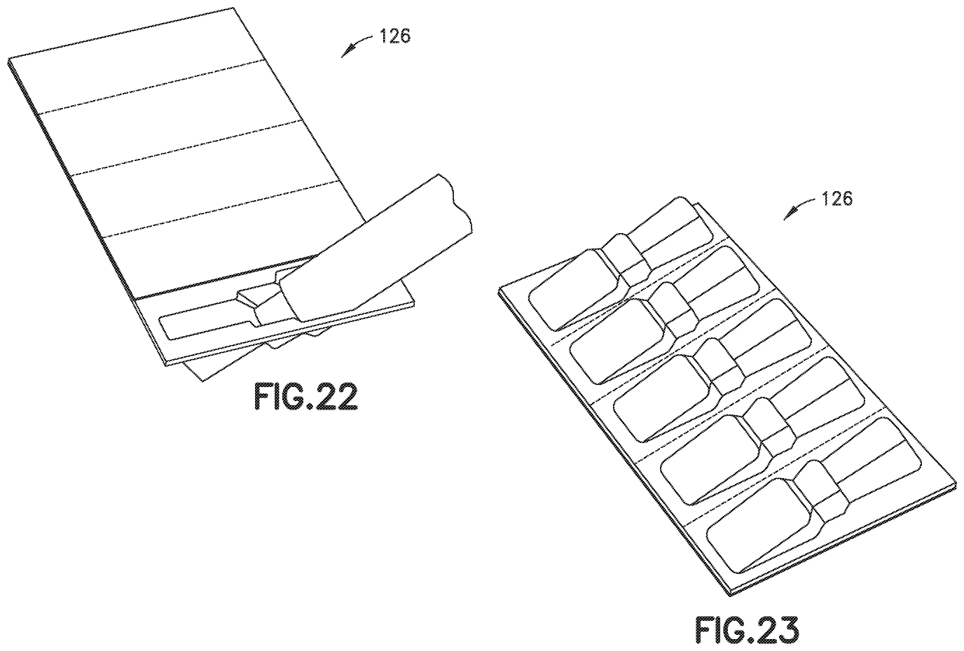

The orientation of the pen needles 10 or other medical sharps can be changed to obtain the desired functionality, while working within the confines of the material limitations. FIGS. 22 and 23 illustrate an angled tray package 126 in which the pen needles 10 or other medical sharps are disposed at an angle, preferably about 45 degrees, to make it easier to attach a medical device, such as pen injector 50 or a syringe to one of the pen needles 10 or medical sharps. Features, such as internal splines, can be formed into these cavities to allow for ease of presentation of the pen needle to the user for assembly onto the pen injector. Preferably, the angled tray package 126 has perforations separating adjacent pen needles 10, so that individual packaged pen needles or smaller arrays can be easily formed for the user's convenience.

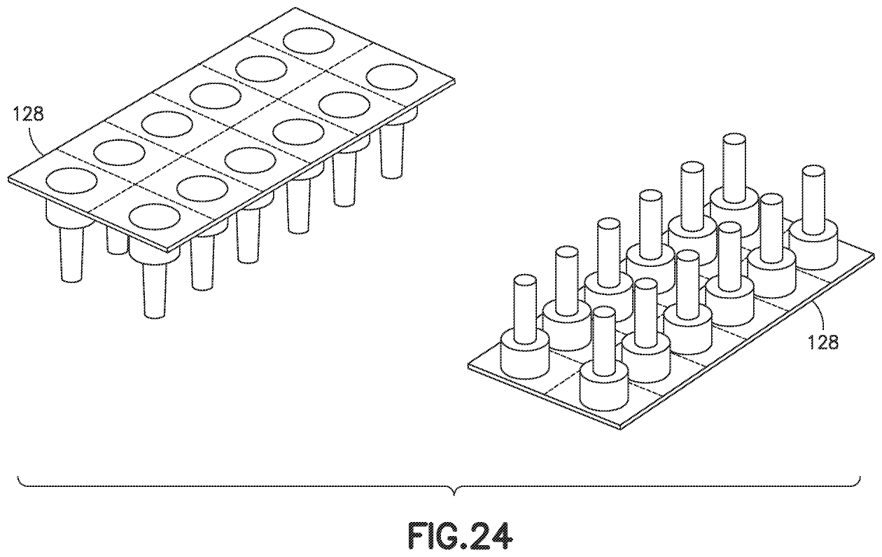

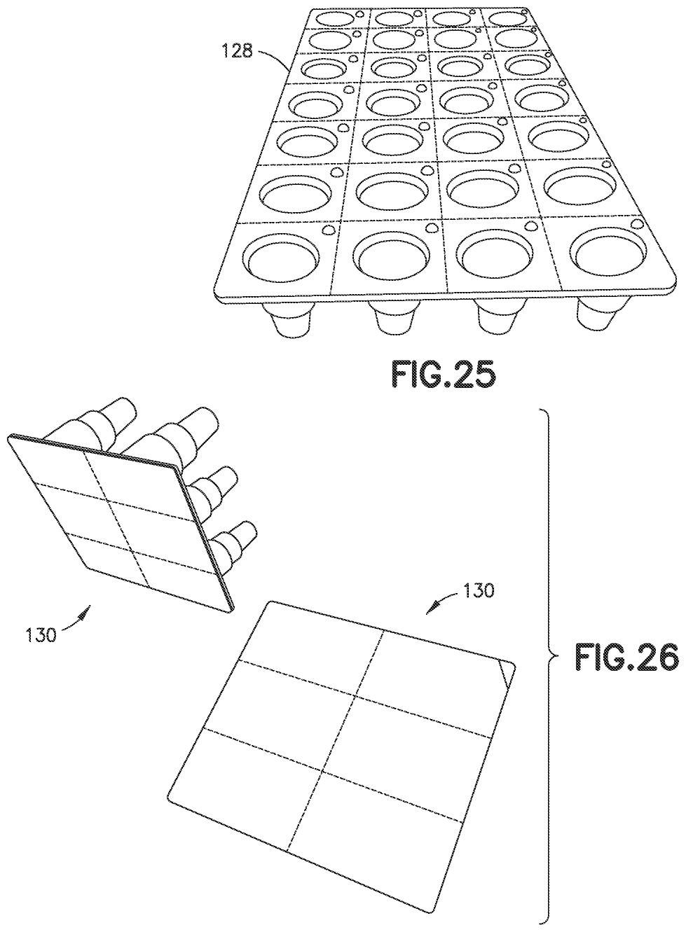

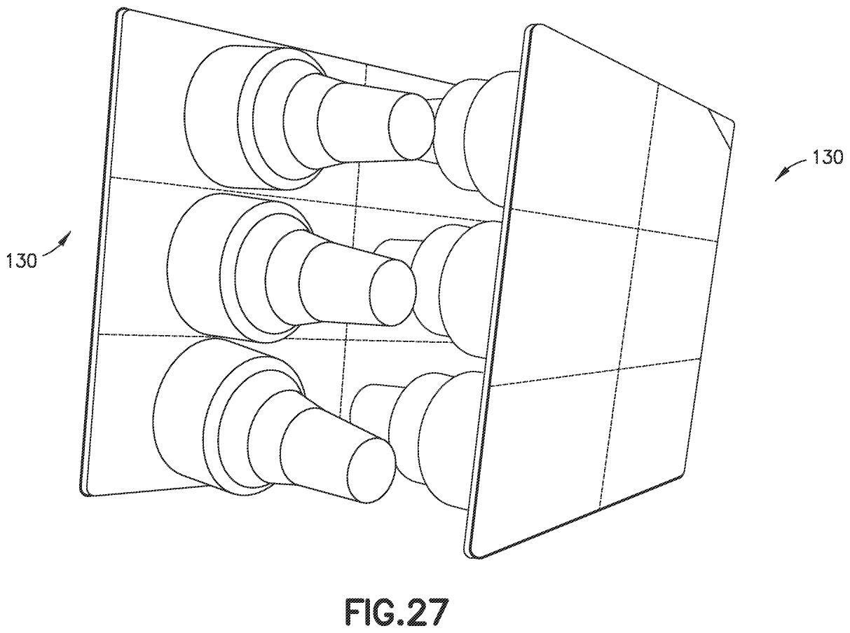

FIGS. 24-27 illustrate vertically oriented trays 128 and vertically oriented tray packages 130 (with a sterility barrier applied) that vertically house an array of pen needles 10 or other medical sharps to make it easier to attach a medical device, such as pen injector 50 or a syringe to one of the pen needles 10 or medical sharps. The vertically oriented trays 128 and vertically oriented tray packages 130 allow for configuration for a large count offering or a small count offering as shown in FIGS. 24-27. Preferably, the vertically oriented trays 128 and vertically oriented tray packages 130 have perforations separating adjacent pen needles 10, so that individual packaged pen needles or smaller arrays can be easily formed for the user's convenience.

The thermoformed tray embodiments utilize substantially less plastic, both in the form factor that houses the pen needle (in comparison, for example, to outer shield 29) and in the size of the sterility barrier, which can be a high fraction of the total cost of the finished package. In addition, because the packing density of the pen needles or medical sharps is higher, similar to the packages 116, the same number of pen needles can be packaged into a smaller box or other outer packaging, thereby reducing transportation and storage costs, for both manufacturers and the retail pharmacies carrying the product on their shelves.

Current sterility barriers, like tear drop cover or label 32, are multi-laminate metallic foils with a paper component for printing. Different layers of the multi-laminate structure provide different functionality, such as components that preferentially melt and cause adhesion onto the outer shield 29, or a layer for printing.

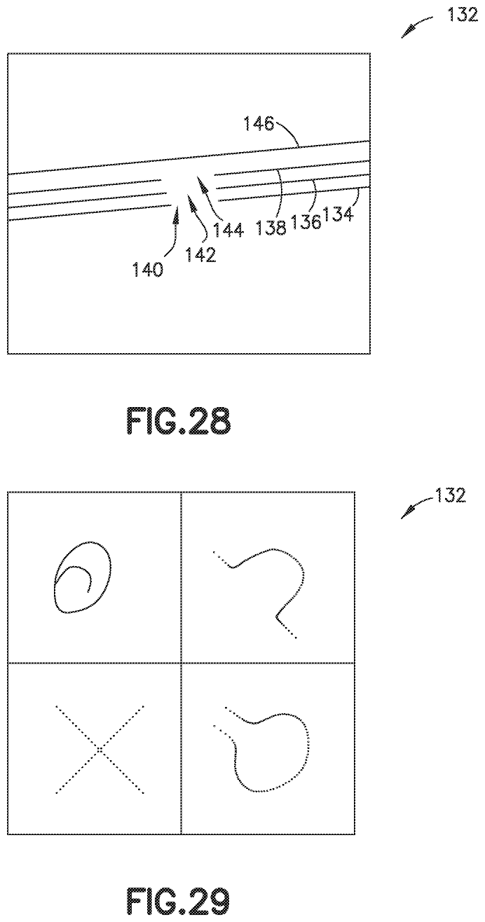

A multi-laminate sterility barrier 132 can be manufactured to be pre-weakened by perforating specific layers prior to lamination, such as shown in FIG. 28, which is a partial close-up side cross-sectional view of the sterility barrier layers depicted with space between the layers for explanatory purposes. For example, sub-layers 134, 136, and 138 of the multi-laminate sterility barrier 132 can have respective perforations 140, 142, 144 therein, while top layer 146 has no such perforations. As another example, one or more of the sublayers 134, 136, and 138 can be formed without perforations. The layers can be paper, foil, or a combination. For example, the top layer 146 can be foil and the remaining layers 134, 136, and 138 can be paper. Other combinations can also be formed. Preferably, the perforations are formed prior to lamination. Such a manufacturing method makes it easier to open the sterility barrier 132. For example, the sterility barrier 132 can be used use in a punch-through fashion instead of having to peel it off the outer cover. In such embodiments, multiple geometries, such as those shown in FIG. 29 for pre-weakening can be employed.

Although embodiments of the present invention have been described in relation to pen needles, it will be understood by one skilled in the art that other medical needles and cannulas with hubs can be packaged in the same or similar way.

The present invention is not limited to use with sharpened medical cannulas such as pen needles and syringe needles. For example, blunt cannulas of the type used to inject medicaments into IV lines through split-septum injection sites can also be packaged as disclosed herein.

Although only a few embodiments of the present invention have been shown and described, the present invention is not limited to the described embodiments. Instead, it will be appreciated by those skilled in the art that changes may be made to these embodiments without departing from the principles and spirit of the invention. Any of the embodiments and/or elements disclosed herein may be combined with one another to form various additional embodiments not specifically disclosed, as long as they do not contradict each other. It is particularly noted that those skilled in the art can readily combine the various technical aspects of the various elements of the various exemplary embodiments that have been described above in numerous other ways, all of which are considered to be within the scope of the invention, which is defined by the appended claims and their equivalents.

1. A package for a medical cannula, comprising:
a plurality of medical cannula outer shields, the outer shields each being configured for receiving a medical cannula therein; and
a web having a plurality of holes therethrough corresponding to the plurality of outer shields, each of the corresponding plurality of holes retaining one of the plurality of medical cannula outer shields therein.

2. The package according to claim 1, wherein while retaining the outer shields in the web, the outer shields are exposed on opposing sides of the web.

3. The package according to claim 1, wherein the web holes have corresponding openings on opposing sides of the web.

4. The package according to claim 1, further comprising:
a plurality of medical cannulas corresponding to the plurality of outer shields, each outer shield housing a medical cannula; and
a removable sterility barrier sealing each medical cannula in one of the outer shields.

5. The package according to claim 4, wherein the sterility barrier is removably attached to each outer shield.

6. The package according to claim 4, wherein the sterility barrier is removably attached to the web.

7. The package according to claim 1, wherein a proximal flange of each outer shield is disposed proximal to the web and a portion of each outer shield is disposed distally of the web.

8. The package according to claim 1, wherein a proximal flange of each outer shield is flush with a proximal surface of the web and a portion of each outer shield is disposed distally of the web.

9. The package according to claim 1, wherein a proximal flange of each outer shield is disposed distally of a proximal surface of the web and a portion of each outer shield is disposed distally of the web.

10. The package according to claim 1, wherein the web comprises a plurality of web units, each web unit comprising a web flange surrounding one of the plurality of holes.

11. The package according to claim 10, wherein each web flange comprises a proximal flange and a recessed flange recessed from a proximal surface of the proximal flange, the recessed flange being configured to receive a flange of one of the plurality of outer shields.

12. The package according to claim 10, wherein the web is perforated between adjacent web units.

13. The package according to claim 10, wherein each web unit comprises one marking of a series of markings, the series of markings collectively signifying different injection sites to aid rotation of injection sites.

14. The package according to claim 10, the package further comprising:
a plurality of medical cannulas corresponding to the plurality of outer shields, each outer shield housing a medical cannula; and
a plurality of removable sterility barriers corresponding to the plurality of medical cannulas, each sterility barrier sealing a medical cannula in one of the outer shields;
wherein at least one of each sterility barrier and each web unit comprises one marking of a series of markings, the series of markings collectively signifying different injection sites to aid rotation of injection sites.

15. The package according to claim 14, wherein the markings are disposed on the sterility barriers.

16. The package according to claim 14, wherein the markings are disposed on the sterility barriers and the web units.

17. A method of packaging a plurality of medical cannulas, comprising:
providing a plurality of medical cannula outer shields, each having an opening therein, the outer shields each being configured for receiving one of the plurality of medical cannulas therein;
respectively inserting the plurality of medical cannulas into the plurality of outer shields;
providing a web having a plurality of holes therethrough corresponding to the plurality of outer shields,
respectively inserting the plurality of outer shields into the corresponding plurality of holes to secure the outer shields in the web; and
providing a sterility barrier for the outer shields with the medical cannula disposed therein.

18. The method according to claim 17, wherein the sterility barrier is removably attached to each outer shield.

19. The method according to claim 17, wherein the sterility barrier is removably attached to the web.

20. The method according to claim 17, wherein a proximal flange of each outer shield is disposed proximal to the web and a portion of each outer shield is disposed distally of the web.

21. The method according to claim 17, wherein a proximal flange of each outer shield is flush with a proximal surface of the web and a portion of each outer shield is disposed distally of the web.

22. A laminated sterility barrier for covering an opening of a container, comprising:
a top layer that is not perforated; and
one or more sub-layers under the top layer, a bottom sublayer being configured for contacting the container, at least one of the sub-layers having perforations therein in a pattern to pre-weaken the sterility barrier for punch through opening and removal of an item sealed in the container by the sterility barrier.
